(19) United States
(12) Patent Application Publication
(10) Pub. No.: US 2010/0067771 A1
(43) Pub. Date: Mar. 18, 2010

(54) ENERGY RESOLVED IMAGING

(75) Inventors: Hannes Dahnke, Hamburg (DE); Michael Grass, Hamburg (DE); Emiel Peeters, Eindhoven (NL)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/516,601

(22) PCT Filed: Nov. 13, 2007
(86) PCT No.: PCT/IB07/54611

§ 371 Date: May 28, 2009

Related U.S. Application Data
(60) Provisional application No. 60/867,859, filed on Nov. 30, 2006.

(51) Int. Cl. G06K 9/00; G01N 23/04; C12N 5/00

(52) U.S. Cl. 382/132; 378/62; 435/402

(57) ABSTRACT

A medical imaging method includes energy-resolving x-ray projection data indicative of a contrast labeled scaffold seeded with biological cells for growing tissue and reconstructing the energy-resolved projection data to generate energy-resolved image data indicative of the contrast labeled scaffold.

The present application generally relates to imaging. While it finds particular application to imaging contrast agent doped scaffolds within a human, it also relates to other medical and non-medical applications in which it is desirable to distinguish structures having similar imaging contrast properties.

The combination of biological cells, engineered materials, and biochemical factors has been used in tissue engineering to grow tissue, such as cartilage, bone, and blood vessels. One approach includes tactically implanting within the body a biologically compatible supporting structure such as a scaffold or a biodegradable stent that has a favorable microstructure and/or has been seeded with particular biological cells or biological factors. Such a structure provides an environment that facilitates cell growth. Over time, it breaks down and is absorbed by the body, and the newly formed tissue takes over the biological and mechanical function. The structure may be formed from synthetic (e.g., polymers and polyesters) or natural (e.g., proteic and polysaccharidic materials) material.

A particular application includes growing cartilage in the knee. In one instance, a cartilage scaffold is seeded with appropriate cells and formed to provide interim mechanical and structural support within the knee. The scaffold is implanted and degrades at a rate to provide such support until the cartilage cells grow and form cartilage tissue that is able to provide the support by itself. Less than desired results may occur if the scaffold is not suitably positioned, the cell growth rate or the scaffold degradation rate is not within a desired range, or the newly grown tissue does not have the desired mechanical properties. As a consequence, it is often desirable to monitor the initial position of the scaffold, subsequent positioning of the scaffold, scaffold degradation, new tissue growth, and the mechanical properties of the new tissue. During therapy, it is desirable to know the rate of tissue growth and scaffold degradation in order to control the stress the knee should undergo during the different phases of the healing process.

One approach to monitoring such characteristics includes periodically imaging the region in which the scaffold is implanted and comparing images generated from data acquired at different times. However, in some instances the scaffold and the tissue of interest are relatively small in size and have similar imaging contrast properties. In other instances, the scaffold is not visible. As a result, the ability to monitor scaffold positioning and degradation and tissue formation via imaging techniques may be less than desired.

Aspects of the present application address the above-referenced matters and others.

According to one aspect, a medical imaging method includes energy-resolving x-ray projection data indicative of a contrast labeled scaffold seeded with biological cells for growing tissue and reconstructing the energy-resolved projection data to generate energy-resolved image data indicative of the contrast labeled scaffold.

According to another aspect, a computer readable storage medium containing instructions which, when executed by a computer, cause the computer to carry out the steps of energy-resolving x-ray projection data indicative of a contrast labeled scaffold seeded with biological cells for growing tissue and reconstructing the energy-resolved projection data to generate energy-resolved image data indicative of the contrast labeled scaffold.

According to another aspect, a medical imaging system includes an energy-resolving detector that detects energy within an energy range and produces energy-resolved projection data, a source that emits polyenergetic radiation that travels through an examination region and an object disposed therein that includes a cell growth supporting structure doped with a contrast agent that corresponds to the energy range of the detector, and a reconstructor that generates energy-resolved image data indicative of the contrast agent from the energy-resolved projection data.

According to another aspect, a system includes a means for labeling a scaffold with a contrast agent, a means for imaging the scaffold to acquire energy-resolved x-ray projection data indicative of the contrast labeled scaffold, and a means for reconstructing the image data to generate energy-resolved x-ray image data indicative of the contrast labeled scaffold.

According to another aspect, a cell growth supporting structure is doped with an element having properties that enhance the image contrast of the cell growth supporting structure relative to surrounding structure for a medical imaging application.

Still further aspects of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

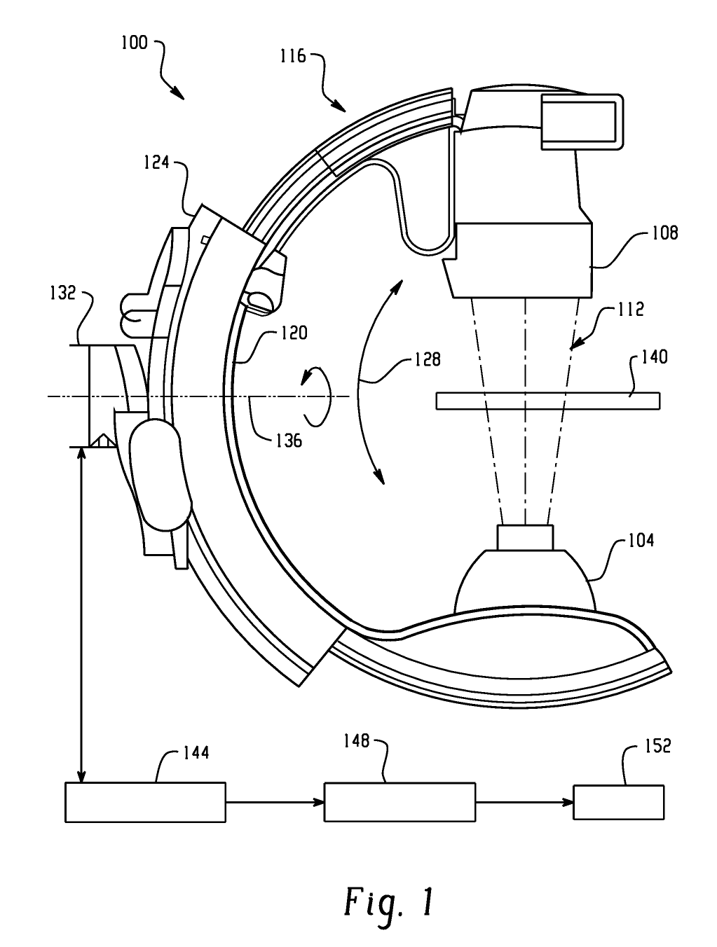

FIG. 1 illustrates an exemplary imaging system.

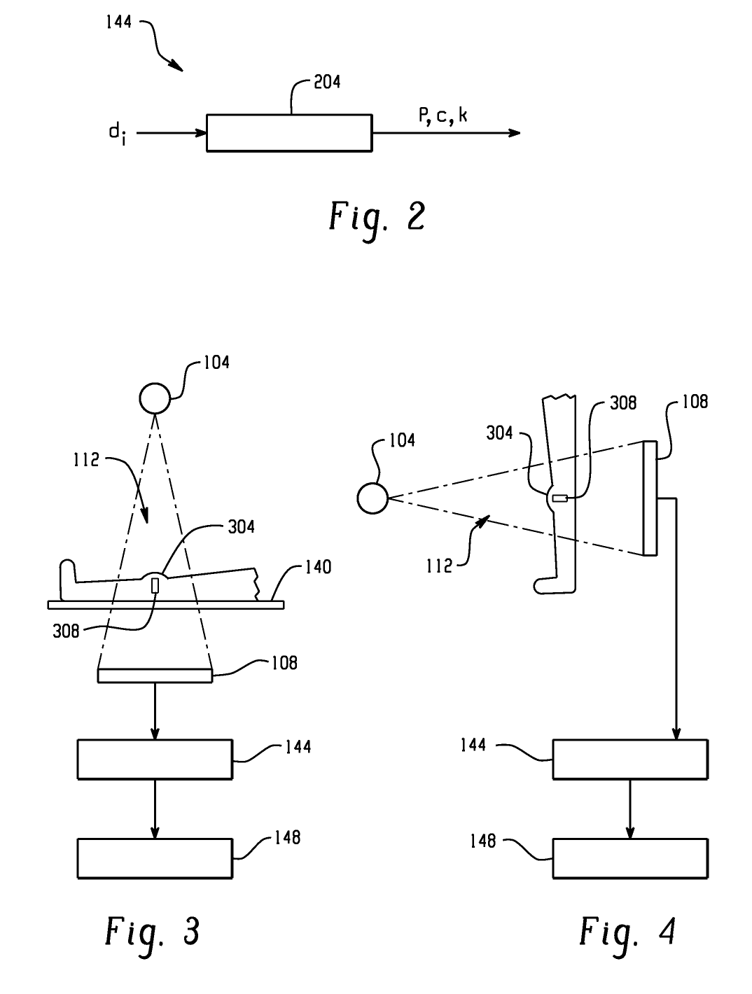

FIG. 2 illustrates an exemplary pre-processor.

FIGS. 3 and 4 illustrate an exemplary pre-processor.

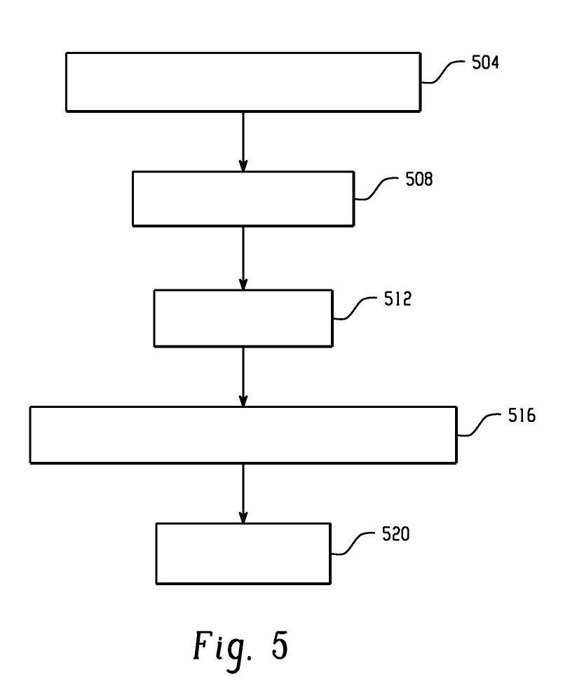

FIG. 5 illustrates an exemplary imaging method.

With reference to FIG. 1, an x-ray imaging apparatus 100 includes an x-ray tube or source 104 and an x-ray sensitive detector 108. The x-ray source 104 generates and emits radiation that traverses an examination region 112 and illuminates the detector 108.

The detector 108 includes a matrix of x-ray radiation sensors or detector pixels. A suitable detector 108 includes a flat panel detector. In the illustrated embodiment, the detector 108 measures the energy of incident photons and counts the number of incident photons within each of a plurality of energy ranges or bins. The energy-resolving detector may alternatively be implemented using multiple scintillation or direct conversion detectors, or other energy-resolving techniques, either alone or in combination. The detector 108 converts the detected energy into electrical signals indicative of the detected energy to generate projection data.

Both the source 104 and the detector 108 are suspended by a moveable C-shaped portion of a support arm 116. The source 104 and the detector 108 move with the support arm 116 and are spatially oriented thereon with respect to each other so that radiation emitted by the source 104 traverses the examination region 112 and strikes the detector 108. The support arm 116 has a displaceable member 120 that displaces within an arc-shaped sleeve member 124. The displaceable member 120 displaces along first and second directions 128, which rotates the source 104 and the detector 108 about the examination region 112. The sleeve member 124 is pivotably coupled to a structure 132 and pivots about a pivot axis 136. Pivoting the sleeve member 124 about the pivot axis 136 moves the support arm 116 through an angular distance so that the source 104 and the detector 108 rotate about the pivot axis 136 and the examination region 112.

The structure 132 is operatively connected to a wall, ceiling, floor, stationary device, mobile device or other support. In one instance, this connection includes one or more intermediate structures that pivot, rotate or translate along a corresponding axis. Such pivoting, rotating, and translating motion provides for multiple degrees of freedom to variously orient the source 104 and the detector 108 about the examination region 112. In one instance, advantageously pivoting, rotating, and translating moveable components moves them in coordination with each other to rotate the source 104 and the detector 108 about the examination region 112 so that x-ray projections are acquired from plurality of different angular positions. The angular extent of the data acquisition is such that projections are obtained over at least one hundred and eighty (180) degrees plus a fan angle. Acquiring data over such an angular extent provides a complete set of data for a three-dimensional rotational x-ray reconstruction.

A patient support 140 supports an object, a patient, or other subject in the examination region 112. The patient support 140 is movable so as to guide the object, patient or other subject within the examination region. In some applications, the support member 140 is moved outside of the examination region (112), and the subject is positioned within the examination region 112 without the support member 140.

An energy pre-processor 144 processes the projection data to provide projection data having a desired spectral characteristic. The inputs to the energy pre-processor 144 include energy the resolved detector signals indicative of energy detected in the energy range of the detector 108.

A reconstructor 148 reconstructs the projection data to generate image data. Image data corresponding to a single x-ray projection is processed to generate a two-dimensional image, and image data corresponding to multiple projections acquired at different angular positions are processed to generate a three-dimensional image. In one instance, the image is generated using a quantitative technique in which the energy measurements for a particular energy bin(s) are processed to show a relative concentration of detected photons having an energy within the energy range of the bin in the image. In another instance, a combination of the attenuation coefficients and energy selection is used to label a desired material within the image.

A general purpose computer serves as an operator console 152. The console 152 includes a human readable output device such as a monitor or display and an input device such as a keyboard and mouse. Software resident on the console 152 allows the operator to control the scanner 100. In one instance, such control includes selecting a scan protocol, adjusting scan parameters, initiating, pausing and terminating scanning, and otherwise interacting with the scanner 100, e.g., through a graphical user interface (GUI).

As noted above, the energy pre-processor 144 processes the projection data from the detector 108 to provide projection data having a desired spectral characteristic. In one implementation, and with reference to FIG. 2, the pre-processor 144 employs a k-edge imaging technique to generate projection data indicative of a contrast agent or other substance present in the subject. The inputs to the energy pre-processor 144 include energy resolved detector signals d₁, d₂ . . . dᵢ indicative of energy detected in a plurality of energy ranges or bins. In the case of k-edge detection, i is preferably greater than or equal to two (2). The detection signals dᵢ exhibit a spectral sensitivity Dᵢ(E) of the i-th energy bin or range bᵢ. Furthermore, the emission spectrum T(E) of the polychromatic radiation source 104 is generally known.

In one implementation, a modeling unit 204 models the subject as a combination of the photo-electric effect with spectrum P(E), the Compton effect with spectrum C(E), and the substance (e.g., a contrast medium) with a k-edge in an energy range of interest and a spectrum K(E). The density length product for each of the components, in particular that of the photo-effect component p, the Compton effect component c, and the k-edge component k, in each detection signal dᵢ can be modeled as a discrete linear system according to the relationship:

dᵢ=∫dE·T(E)·Dᵢ(E)·(p·P(E)+c·C(E)+k·K(E)).

Where at least three detection signals d₁, d₂, d₃ are available for at least three energy ranges or bins b₁, b₂, b₃, a system of at least three equations is formed having three unknowns, which can thus be solved with known numerical methods in a calculation unit 208. The results, in particular the components p, c, and k, can then be used alone or in combination to reconstruct images of the desired component using conventional reconstruction methods.

While three energy ranges or bins bᵢ are generally sufficient to determine the components p, c, and k, improved sensitivity and noise robustness may generally be obtained by improving the energy resolution of the input signal, for example by increasing the number of ranges or bins bᵢ.

FIGS. 3 and 4 provide two examples in which the system 100 images an object doped with a contrast agent having characteristic energy corresponding to an energy range of the detector 108. In this example, the object is a cell growth supporting structure such as a scaffold 308 disposed within a human knee 304. The scaffold 308 is formed from an artificial (or synthetic) or a natural material or a combination thereof and is seeded with suitable biological cells that grow to form a tissue of interest, which in this example is knee cartilage.

According to a one embodiment, the natural polymer forming the scaffold is agarose, alginate, hyaluronic acid, chitosan, collagen, gelatin, silk or combinations thereof. Synthetic materials for the formation of scaffolds include poly (caprolactone), poly(glycolic acid), poly(L-lactic acid), Poly D, L-lactic-co-glycolic acid), poly(propylene fumarate), poly(orthoester), poly(anhydride), poly(maleic acid), poly(p-dioxanone, poly(trimethylen carbonate), poly(3-hydroxybutyrate), poly(3-hydroxyvalorate) and copolymers thereof. The natural or synthetic materials are processed such that a scaffold is formed with a macro- and microstructural properties such as spatial form, mechanical strength, density, porosity, pore size, pore distribution and pore interconnectivity, favorable for the growth of biological cells. Processing methods include, solvent casting/particulate leaching, gas foaming, fiber bonding, phase separation, melt molding, emulsion freeze-drying and various solid freeform fabrication techniques such as three-t-dimensional bioplotting, robotic micro-assembly. It is to be appreciated that in another example the object is a resorbable stent or the like.

Various techniques are used to dope or treat the scaffold 308 with the contrast agent. For example, when forming a scaffold with an artificial material, the contrast agent can be included and synthesized with the artificial material to concurrently form and label the scaffold. In this way, the contrast agent can be linked to the artificial material through covalent or ionic bonds. The contract agent can also be physically entrapped (or dissolved) in the arftificial material during the formation of the scaffold without covalent or ionic linkages. Alternatively, the contrast agent can be grafted to such a scaffold after the scaffold is formed. Grafting may be achieved via a bonding technique such as chemical bonding in which elements of the contrast agent bond with elements of the scaffold. When forming a scaffold from a natural material, the contrast agent is grafted to the scaffold as described above or entrapped in the natural scaffold materials during scaffold formation.

The scaffold 308 is labeled with gadolinium (Gd). Gadolinium (Z=64) has a K-edge at about fifty (50) kiloelectron volt (keV), which lies within the diagnostic energy spectrum, which generally is from about forty (30) keV to about one hundred seventy (170) keV. A photoelectric interaction between a photon emitted by the source 104 having sufficient energy (having energy equal to or greater than the k-shell binding energy of gadolinium) and a k-shell electron of the gadolinium results in the absorption of the photon and ejection of a photoelectron, leaving a vacant hole in the k-shell.

By labeling the scaffold 308 with gadolinium the corresponding binned energy data can be used to enhance the image contrast of the scaffold 308 within the image relative to the surrounding biological tissue. Generally, biological tissues includes mostly low atomic number elements (e.g., hydrogen (H), Z=1; carbon (C), Z=6; nitrogen (N), Z=7; and oxygen (O), Z=8), which have relatively lower k-shell binding energies and, thus, yield relatively few characteristic x-rays. In addition, for photoelectric absorption in tissues, the characteristic energy generally is totally absorbed by the object and is not detected by the detector 108. Thus, the image data corresponding to this energy bin is largely indicative of the gadolinium.

In operation, the scaffold 308 generally is imaged in a non-weight bearing or resting position as shown in FIG. 3 or in a weight bearing or stressed position as shown in FIG. 4. The resulting image data is reflective of a state of the contrast agent and, thus, the scaffold 308 at the time it was imaged. This data can be used to spatially locate the contrast labeled scaffold 308 within the knee. By comparison of the image data or image(s) under non-weight bearing conditions with the image data under weight bearing conditions information can be derived on the mechanical properties of the mechanical properties of the scaffold. By acquiring data during one or more different time intervals, the image data or image(s) generated therefrom is representative of the state of the scaffold 308 at different moments in time. This data can indicate whether the scaffold 308 has moved to a different position. Additionally or alternatively, this data provides information about scaffold characteristics over time. For instance, the scaffold 308 degrades over time as the cells thereon grow to form the cartilage. By imaging the scaffold 308 at different times, degradation of the scaffold 308 can be tracked over time. Since scaffold degradation is related to cell or tissue growth, the amount of scaffold degradation between different time periods can also be used to characterize cell growth during the different time periods.

With reference to FIG. 5, operation will now be described in relation to exemplary contrast enhanced imaging method.

At reference numeral 504, an object including a scaffold doped with a contrast agent having desired properties is suitably positioned within an examination region.

At 508, a number of projections are acquired at different angular locations around the object to provide a complete set of energy-resolved projection data for reconstruction.

At 512, the projection data is reconstructed to generate image data. As described above, images may be generated from the image data, and the images are used to spatially locate the contrast agent and, hence, the scaffold 308 within the object.

At 516, if desired, another set of projections is obtained during a subsequent time period and generate image data therefrom. It is to be appreciated that N sets of projection data corresponding to M different time periods can be acquired.

At 520, image data corresponding to different periods is used to monitor the spatial position of the scaffold 308 in the object, scaffold degradation, and cell growth over time.

Variations are contemplated.

In the illustrated embodiment, the scaffold 308 is labeled with gadolinium. In other embodiments, the scaffold is alternatively labeled with iodine (I), Z=53, barium (Ba), Z=56, lanthanum (La), Z=57, gold (AU), Z=79, or other elements that have a desirable k-shell binding energy or eject photons having desirable characteristic energy.

The technique described herein is also amenable to other imaging modalities, including, but not limited to, computed tomography (CT) and magnetic resonance imaging (MRI). With a CT system, the scaffold 308 is similarly labeled with gadolinium, iodine, barium, lanthanum, gold, etc. One advantage of using a CT based system is improved contrast resolution. With a MRI system, the scaffold 308 is labeled with gadolinium, iron oxide, or other materials with desired properties.

The spectral information may be obtained other than through the use of the energy-resolving detector 108. For example, an x-ray source(s) that generates radiation having desired spectral characteristics and/or time varying or other filters that selectively harden or otherwise alter the spectral characteristics of the radiation may also be used. A method that describes energy resolved reconstruction with iterative methods from projections acquired with different spectra but without an energy resolving detector is described in WO 03/071483 A2 by Fessler et al.

In addition, other processing techniques that identify a substance of interest such as a contrast agent or otherwise provide a desired material separation may also be implemented. Depending on the technique, data indicative of three (3) energy ranges or bins may be used, for example, where it is desirable to separate components of the acquired projection data or where it is desired to interpolate the energy resolved data.

In an alternative implementation, the energy pre-processor 144 may be omitted. In such an implementation, the reconstructor 148 may operate directly on the energy resolved projection data. An energy-based post processor which operates on the image data may also be used to identify a substance of interest or otherwise provide a desired material separation.

The energy pre-processor 144 and the reconstructor 148 may be implemented by way of computer readable instructions which, when executed by a computer processor(s), cause the processor(s) to carry out the described techniques. In such a case, the instructions are stored in a computer readable storage medium associated with or otherwise accessible to the relevant computer. The described techniques need not be performed concurrently with the data acquisition. They may also be performed using a computer (or computers), which are associated with the scanner 100; they may also be located remotely from the scanner 100 and access the relevant data over a suitable communications network such as a HIS/RIS system, PACS system, the internet, or the like.

While the above description has focused on imaging the knee, the described techniques may also be used in connection with tissue other than knee cartilage such as cartilage corresponding to other anatomy, bone, and blood vessels.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A method, comprising:
energy-resolving x-ray projection data indicative of a contrast labeled scaffold seeded with biological cells for growing tissue; and
reconstructing the energy-resolved projection data to generate energy-resolved image data indicative of the contrast labeled scaffold.

2. The method of claim 1, wherein the contrast agent is one of iodine, gadolinium barium, lanthanum, and gold.

3. The method of claim 1, where the scaffold includes a synthetic or a biological material.

4. The method of claim 1, wherein the biological cells grow to form biological tissue.

5. (canceled)

6. The method of claim 1, further including reconstructing the image data to generate a three-dimensional image of the scaffold.

7. (canceled)

8. (canceled)

9. The method of claim 1, further including rotating an x-ray source and a detector around the scaffold, wherein the detector detects radiation emitted by the source that traverses an examination region and generates projection data indicative of the detected radiation.

10. The method of claim 1, wherein the detector is an energy-resolving detector that detects radiation within a plurality of energy ranges.

11. The method of claim 1, wherein the source emits radiation having a desired spectral characteristic.

12. A computer readable storage medium containing instructions which, when executed by a computer, cause the computer to carry out the method of claim 1.

13. The computer readable storage medium of claim 12, wherein the computer is a console of an x-ray system, wherein the system includes:
a radiation source that emits x-rays that traverse an examination region;
a detector that emitted detects radiation that traverses the examination region and generates projection data indicative thereof;
a C-shaped support arm to which the source and the detector are operatively coupled, wherein angularly moving the arm rotates source and the detector about the scaffold; and
a component that energy resolves the projection data.

14. A medical imaging system, comprising:
an energy-resolving detector that detects energy within an energy range;
a source that emits energy that travels through an examination region and an object disposed therein that includes a cell growth supporting structure doped with a contrast agent that corresponds to the energy range of the detector; and
a reconstructor that generates energy-resolved data indicative of the contrast agent doped structure from the detected energy.

15. (canceled)

16. (canceled)

17. The system of claim 14, wherein the supporting structure is seeded with cells for growing one of cartilage, bone, and a blood vessel.

18. The system of claim 14, wherein a k-edge technique is employed to generate energy-resolved projection data.

19. The system of claim 14, wherein the source and detector rotate about the object over at least one hundred and eighty degrees plus a fan angle and projection data are acquired at a plurality of different angular positions over the angular extent.

20. The system of claim 19, wherein image data generated from the projection data is used to generate a three-dimensional image of the contrast agent.

21. The system of claim 14, wherein an image is generated from the image data, and the image indicates a spatial location of the contrast agent doped support structure within the object.

22. The system of claim 14, wherein image data acquired at different times reflects a change of a state of the contrast doped supporting structure.

23. The system of claim 22, wherein the change of state is indicative of support structure degradation.

24. The system of claim 22, wherein the change of state is indicative of movement of the support structure within the object.

25. (canceled)

26. A system, comprising:
means for labeling a scaffold with a contrast agent;
means for imaging the scaffold to acquire energy-resolved x-ray projection data indicative of the contrast labeled scaffold; and
means for reconstructing the image data to generate energy-resolved x-ray image data indicative of the contrast labeled scaffold.

27. A cell growth supporting structure doped with an element having properties that enhance the image contrast of the cell growth supporting structure relative to surrounding structure when imaging with a medical imaging system.

28. The cell growth supporting structure of claim 27, wherein the agent has a k-shell binding energy within an energy spectrum of an x-ray imaging system used to image the cell growth supporting structure.

29. (canceled)

30. The cell growth supporting structure of claim 27, wherein the agent is one of gadolinium, barium, lanthanum, and gold.

31. The cell growth supporting structure of claim 27, wherein the cell growth supporting structure is a scaffold located within a patient.

32. (canceled)
